(19) United States
(12) Patent Application Publication
(10) Pub. No.: US 2006/0228423 A1
(43) Pub. Date: Oct. 12, 2006

(54) Cartilage composition for transplantation

(76) Inventors: Hiroko Yanaga, Fukuoka (JP); Katsu Yanaga, Fukuoka (JP)

Correspondence Address: BIRCH STEWART KOLASCH & BIRCH, PO BOX 747, FALLS CHURCH, VA, 22040-0747, US

(21) Appl. No.: 11/350,929

(22) Filed: Feb. 10, 2006

(30) Foreign Application Priority Data
Apr. 11, 2005 (JP) 2005-113044
Jul. 15, 2005 (JP) 2005-207822

(51) Int. Cl. A61K 35/12

(52) U.S. Cl. 424/572

(57) ABSTRACT

The invention relates to provide a cartilage composition for transplantation which contains a chondrocyte tissue per se formed by culturing chondrocytes; and a method of transplanting the cartilage using the same. The cartilage composition obtained by the invention is free from any risk of viral or bacterial infections and contains normal human chondrocytes or a chondrocyte mass.

### FIELD OF THE INVENTION

This invention relates to a cartilage composition for transplantation adequately usable in repairing a defect in a cartilage or a bone caused by a trauma, congenital malformation, removal of a foreign matter, denaturation due to aging or the like, and a method of transplanting a cartilage using the same.

### BACGROUND OF THE INVENTION

A cartilage tissue differs from other tissues in that it is not merely made up by chondrocytes but rich in fibers and the matrix formed therein. It is known that, in the process of culturing chondrocytes in vitro, the chondrocytes per se produce collagen, aggrecan, proteoglycan, hyaluronic acid, chondroitin sulfate and so on and thus form a so-called cartilage tissue (see, Yasuhiko Oyano, Hone to Nankotsu no Baioroji: Kiso kara Rinsho eno Tenkai, p. 143-149, Kanehara Shuppan, Tokyo (2002); and Mok S S, Masuda K, Hauselmann H J, et al.: Aggrecan synthesized by mature bovine chondrocytes suspended in alginate. Identification of two distinct metabolic matrix pools, J Biol Chem 269:33021-33027(1994)). In transplanting cultured chondrocytes, it has been a practice to employ chondrocytes, which have been separated from fibers and matrix, as such (see, M. Brittberg, New England Journal of Medicine, Vol. 331, No. 14, p. 889-895 (1994), in particular, p. 890, FIG. 1). However, the tissue containing chondrocytes as described above should be disintegrated in this method and, therefore, chondrocytes can be obtained in only a small number of 2.6 to 5×10⁶ (50 to 100 μl in volume) at most, this being not enough for transplantation (see the documents cited above).

In the case where chondrocytes are separated by repeatedly treating them with an enzyme such as trypsin in the subculture before the transplantation, the thus transplanted cartilage shows accelerated dedifferentiation into fibroblasts and the formation of the desired cartilage is inhibited thereby (see, Yasuhiko Oyano, Hone to Nankotsu no Baioroji: Kiso kara Rinsho eno Tenkai, p. 143-149, Kanehara Shuppan (2002)).

In the existing method, use is made of a technique which comprises trypsinizing and separating cultured chondrocytes, seeding the chondrocytes on a template (scaffold) made of an artificial or natural absorbable polymer to thereby induce the production of extracellular matrix by the chondrocytes, then transplanting the chondrocytes into a living (animal) body and thus forming a cartilage (Vacanti C A, Langer R, Schloo B, Synthetic biodegradable polymers seeded with chondrocytes provide a template for new cartilage formation. Plast. Reconstr. Surg. 88:753-759, (1991); and Cao Y L, Vacanti J P, Paige K T, Upton J, Vacanti C A, Transplantation of chondrocytes utilizing a polymer-cell construct to produce tissue-engineered cartilage in the shape of a human ear. Plast. Reconstr. Surg. 100:297-302(1997)). However, this technique suffers from the problem that inflammation occurs upon the absorption of the artificial polymer after being transplanted into the living body and consequently the chondrocytes per se are absorbed in the injured state. Therefore, it is impossible to form a satisfactory cartilage in the affected site after the transplantation by using this method.

Moreover, attempts have been also made to employ various biological components of foreign origins, for example, using fetal bovine serum (FBS) in chondrocyte culture or using bovine collagen as a scaffold (Nobuo Adachi et al., Igaku no Ayumi: Vol. 200, No. 3, 258-259 (2002), using atelocollagen; Yasuhiko Oyano, Hone to Nankotsu no Baioroji: Kiso kara Rinsho eno Tenkai, p. 143-149, Kanehara Shuppan (2002), using collagen, agarose and alginate beads; Vacanti C A, Langer R, Schloo B, Synthetic biodegradable polymers seeded with chondrocytes provide a template for new cartilage formation. Plast. Reconstr. Surg, 88:753-759 (1991); and Cao Y L, Vacanti J P, Paige K T, Upton J, Vacanti C A (1997), Transplantation of chondrocytes utilizing a polymer-cell construct to produce tissue-engineered cartilage in he shape of a human ear. Plast. Reconstr. Surg. 100:297-302). However, the use of such foreign matters has the problem that a transplanted cartilage might be contaminated with various pathogenic viruses, infectious prions or the like, which might lead to an infection.

### SUMMARY OF THE INVENTION

An object of the present invention is to provide a cartilage composition for transplantation and a method of transplanting a cartilage whereby the above-described troubles encountered in the existing methods can be overcome.

That is to say, the inventors have conducted extensive studies on cartilages for transplantation to overcome the troubles encountered in the existing methods and consequently found out that a chondrocyte-containing tissue formed by culturing chondrocytes in vitro can be transplanted as such, thereby completing the present invention.

Thus, the present invention relates to: (1) a cartilage composition for transplantation which contains a chondrocyte tissue per se formed by culturing chondrocytes; and (2) a cartilage composition for transplantation containing a cartilage block which is obtained by forming a cartilage tissue by culturing chondrocytes and then subjecting it to tissue culture.

The present invention further relates to: (3) a cartilage composition for transplantation which contains a cartilage block obtained by carrying out tissue culture in vitro; or (4) a cartilage composition for transplantation which contains a cartilage block obtained by carrying out tissue culture in vivo.

The cartilage composition for transplantation according to the present invention is adequately usable in repairing a defect in a cartilage or a bone in the facial skull, auricle, nose, articulation or the like caused by a trauma, congenital malformation, removal of a foreign matter, denaturation due to aging or the like.

During in vitro cell culture, chondrocytes form a chondrocyte tissue surrounded by the matrix comprising fiber cells, collagen fiber, aggrecan, proteoglycan, hyaluronic acid, chondroitin sulfate and so on. Therefore, these chondrocytes can be mechanically peeled off from a cultivator without resorting to an enzymatic treatment. The thus obtained chondrocytes and the chondrocyte tissue, which is made up of the cartilage matrix produced by the chondrocytes per se, can be used as such as a cartilage composition for transplantation. It has been clarified that by transplanting a cartilage composition containing a chondrocyte tissue per se, a cartilage can be formed soon after the transplantation without causing the dedifferentiation of the chondrocytes into fibroblasts, which is generally observed after transplantation. The transplanted cartilage tissue can be sustained over a longer time than ever before owing to the stem cells contained therein. According to the present invention, about 1 to 5×10³ to 10⁴ chondrocytes are collected from a patient, separated via trypsinization or other methods, and then cultured. Next, the chondrocytes and the matrix produced thereby are collected together and thus a chondrocyte tissue containing about 2 to 6×10⁷ cells can be obtained in the form of 5 to 10 ml of a gel-type solution. Thus, the chondrocytes can be obtained in a sufficient amount for transplantation and the problem of the deficient quantity occurring in the existing techniques can be solved.

Moreover, it was clarified that addition of fibrin as a support or a scaffold for the cells to a chondrocyte tissue followed by transplantation into a living body made possible to form a cartilage block in such a large size that had not been obtained hitherto. This is favorable because it avoids unexpected side effects that employing autologous fibrin might cause.

In the existing transplantation method, it has been a practice to directly transplant chondrocytes into a desired site (affected site). In this method, however, the transplanted chondrocytes form a matured cartilage one to three months after the transplantation and, therefore, a cartilage in a desired shape or size cannot be obtained. To cope with this problem, a chondrocyte tissue, which has been formed by cell culture, is further subjected to tissue culture to form a cartilage block. After adequately modifying the shape and size, the cartilage block is transplanted into a target site (affected site). As a result, it becomes possible for the first time to control the shape of the transplanted cartilage block so as to give, for example, an auricle-shaped graft in a desired size.

The tissue culture of the chondrocyte tissue that has been formed by culturing chondrocytes can be carried out by culturing the tissue in vitro in a liquid medium for tissue culture suitable for the proliferation of the tissue, or by transplanting the chondrocyte tissue into another site of the living body (for example, the abdomen wherein a wound is less invisible) and then forming a cartilage block therein (i.e., in vivo culture).

It is desirable from the viewpoint of safety that a cartilage composition for transplantation originates in humans and comprises autologous tissues. It is also desirable to avoid using a nonhuman animal-origin foreign protein or an artificial material in the process of preparing chondrocytes to be transplanted into the human body. This is because such a nonhuman animal-origin foreign protein or an artificial material might stimulate immunological reactions or the like in the living body side and thus induce inflammations. As a result, there arises rejection and thus the transplanted cells are also eliminated from the living body. In addition, the use of foreign matters is always accompanied with a risk of infections with various pathogenic viruses and prions capable of infecting humans.

To avoid rejection and lessen the risk of infections with pathogenic viruses and prions capable of infecting humans caused by various biological materials of foreign origins, it is recommended in the present invention to use autologous chondrocytes, autologous extracellular matrix, autoserum and autologous fibrin (serving as a scaffold for autologous cells). In the case where autologous fibrin cannot be collected, use can be also made of a marketed fibrin preparation approved for medical uses. In addition to fibrin, it is also possible to use as a support a biological material or an artificial product so long as its safety has been confirmed.

By using the cartilage composition for transplantation according to the present invention, a cartilage can be formed soon after the transplantation without causing the dedifferentiation of the chondrocytes into fibroblasts, which is generally observed after transplantation.

Since the cartilage composition for transplantation according to the present invention can be easily obtained in a sufficient amount for transplantation, the problem of the deficient quantity occurring in the existing techniques can be solved thereby.

According to the method which comprises forming a chondrocyte tissue by culturing chondrocytes, further subjecting the obtained chondrocyte tissue to tissue culture to thereby form a cartilage block, adequately modifying the shape and size of the cartilage and then transplanting it into a target site (affected site), it becomes possible for the first time to control the shape of the cartilage block before transplantation so as to give, for example, an auricle-shaped graft. Moreover, chondrocytes can be easily obtained in a sufficient amount for transplantation.

The use of the cartilage composition for transplantation according to the present invention makes it possible to avoid rejections. Moreover, the problems of the infections with pathogenic viruses and prions capable of infecting humans caused by various biological materials of foreign origins can be avoided thereby.

### DESCRIPTIONS OF THE DRAWINGS

FIG. 1 shows a chondrocyte tissue formed by culturing chondrocytes until becoming confluent.

FIG. 2 shows the result of hematoxylin-eosine (HE) staining of the chondrocyte tissue formed after culturing wherein the cells are multilayered and bonded together via the matrix.

FIG. 3 shows the result of immunological staining for type II collagen serving as a molecular marker of chondrocyte tissues wherein the extracellular matrix is stained, thus indicating that the extracellular matrix has formed a cartilage-specific matrix.

FIG. 4 shows the results of the assay of chondrocalcin and alkaline phosphatase in the medium and indicates that these substances are produced with the passage of time.

FIG. 5 shows the result of immunological staining of a chondrocyte tissue collected from a transplantation site 6 months after the transplantation for type II collagen serving as a molecular marker.

FIG. 6 shows the result of toluidine blue staining of a chondrocyte tissue collected from a transplantation site 6 months after the transplantation.

FIG. 7 shows that a cartilage having a sufficient size for forming adult auricle is obtained 6 months after the subcutaneous transplantation in the abdomen by using the method of the present invention.

In FIG. 8A, the cartilage size is shown together with a measure, thus indicating that it has a sufficient size for forming auricle. FIG. 8B shows that the thus formed cartilage shows elasticity upon bending.

FIG. 9 indicates that an auricle-shaped frame can be obtained from a cartilage composition for transplantation made of a cultured cartilage.

### DESCRIPTION OF THE INVENTION

1. Outline of the Chondrocyte Culture

The medium to be used in culturing chondrocytes contains not fetal calf serum (FCS) but human serum, preferably human autoserum. The human serum is obtained by collecting autologous blood together with the chondrocytes, centrifuging the blood to thereby remove blood cell components and dividing the residue into the serum (containing autologous proliferation factors) and autologous fibrin. The autoserum and the autologous fibrin may be separately frozen and thawed before use when needed. To culture the chondrocytes, 10% of human serum (preferably human autoserum) is added to the DME medium. In the case of storing fibrin (preferably autologous fibrin), it is preferably stored in a frozen state at −20 to −196° C. The chondrocytes are cultured in a cultivator. After starting the primary culture, the chondrocytes are subcultured over one or two generations and the obtained chondrocyte tissue (about 5×10⁷ to 1×10⁸ cells/ml) is used in transplantation.

It is preferred that the fibrin mass having been frozen as described above is thawed and then the chondrocyte tissue (preferably about 5×10⁷ to 1×10⁸ cells/ml) is added thereto followed by transplantation. Thus, the chondrocytes are stabilized in fibrin and aggregate together to form a mass. To examine infections with bacteria, mycoplasmas and so on, the medium is subjected to a bacterial/mycoplasma culture test once each week to thereby confirm it is negative. By using autologous tissues throughout the culture process, infections with foreign pathogenic bacteria and unexpected infections with viruses and prions can be prevented.

The cartilage composition for transplantation obtained by the present invention is usable in transplanting and forming any human cartilage tissues including auricular cartilage, nasal septal cartilage, costal cartilage, articular cartilage, intervertebral cartilage, tracheal cartilage or pharyngeal roof.

2. Separation of Chondrocytes

A piece (1×1 cm) of auricular cartilage, costal cartilage or articular cartilage was collected and disinfected with penicillin G (800 u/ml), kanamycin (1 mg/ml) and Van Gieson (2.5 Ug/ml). Then the piece was diced with a surgical knife and allowed to stand at 4° C. overnight in the F-12 medium containing 0.3% of type II collagenase (Worthington Biochemicals). On the next day, the culture medium was shaken at 37° C. for 2 to 4 hours, filtered through a nylon mesh and centrifuged to separate chondrocytes.

3. Medium for Chondrocyte Culture

To culture human chondrocytes, use can be made of publicly known media suitable for culturing chondrocytes. The media may optionally contain a proliferation factor such as hydrocortisone (HC), human bFGF or human IGF-I (Cuevas et. al., Biochem. Biophys. Res. Commun. Vol. 156, 611-618 (1988); and Froger-Gaillard et al., Endocrinol. Vol. 124, 2365-72). As an example of such a medium, the DME(H) medium containing autoserum (preferably about 10%), human recombinant bFGF (preferably not more than 100 ng/ml; KAKEN PHARMACEUTICAL), hydrocortisone (preferably not more than 100 ng/ml) and human recombinant IGF-I (preferably not more than 50 ng/ml; GIBCO) can be cited.

4. Primary Culture

By using the cell fraction obtained above, the chondrocytes were seeded in the above-described medium contained in a flask (bottom area: 75 cm²) at an optimum density of 1×10⁴⁻⁵ cells and cultured in a CO₂ incubator at a CO₂ concentration of 10%. During the culture, the medium was replaced twice a week. As a result, the chondrocytes formed a confluent monolayer within a culture time of 10 to 14 days. The obtained cells were used for the following subculture.

5. Subculture

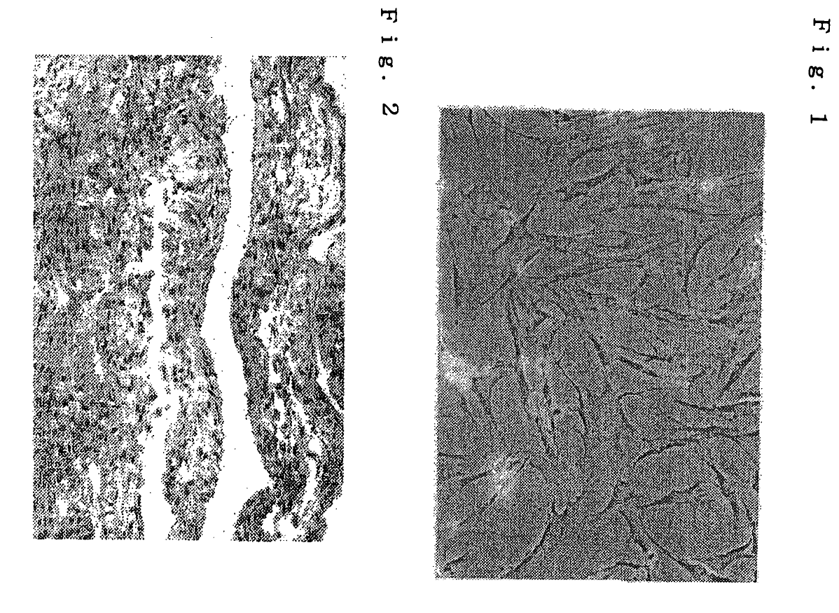

Subculture was carried out by seeding the primary-cultured cells in a flask (bottom area: 175 cm²) at an optimum density of about 1×10⁶ cells and employing the same conditions as in the primary culture. After culturing for 7 days, the cells formed a confluent monolayer (FIG. 1). The obtained cells were employed in the next subculture. As a result, the cell count on the fourth subculture increased about 1000 times, compared with the cell count at the initiation of the subculture. The subcultured chondrocytes were further subjected to cell culture at the preferred density of about 1×10⁶ cells/cm². Thus, a chondrocyte tissue was formed after 3 to 4 weeks.

6. Confirmation of the Formation of Chondrocyte Tissue in Cell Culture

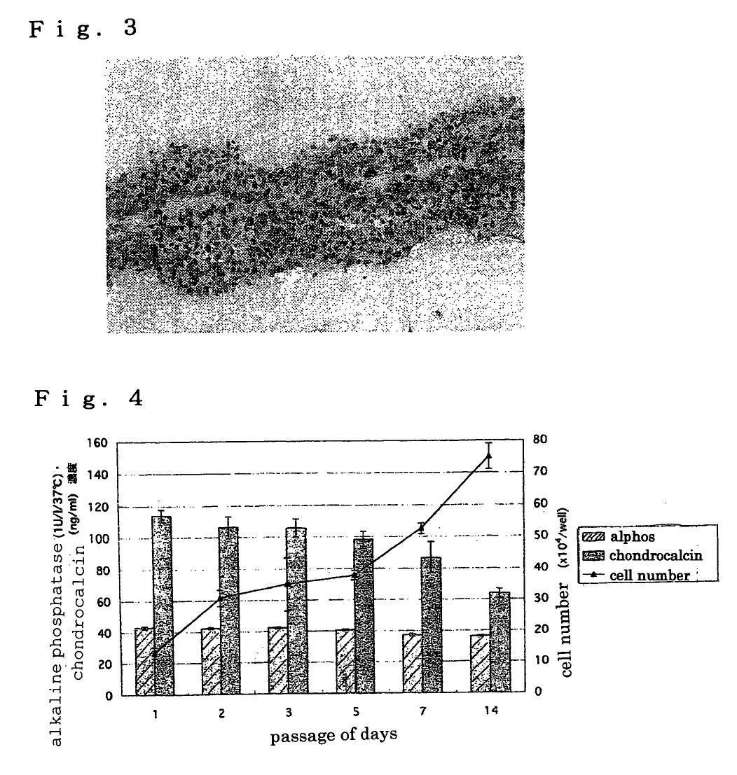

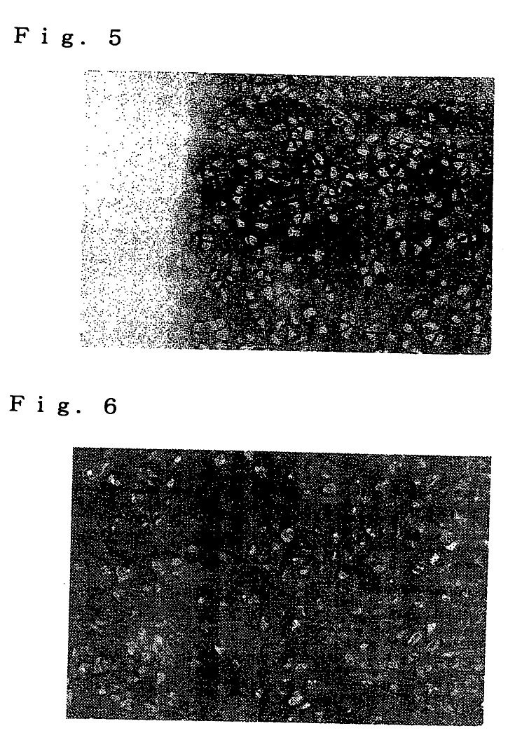

The formation of the chondrocyte tissue was confirmed based on the following facts: (1) when the chondrocyte mass was stained with hematoxylin-eosine (HE), it was observed that the cells were multilayered and bonded together via the matrix (FIG. 2); (2) when the cells were immunologically stained for type II collagen serving as a molecular marker of chondrocyte tissues, the extracellular matrix was stained, thus indicating that the extracellular matrix formed cartilage-specific collagen (FIG. 3); and (3) when chondrocalcin (a C-terminal peptide excised and separated from type II collagen) in the medium was assayed, it was found out that chondrocalcin had been produced (FIG. 4). These facts indicated that type II collagen produced by the chondrocytes had been digested and released into the medium to thereby form the chondrocyte tissue. When alkaline phosphatase (ALP) in the medium was assayed, it was further found out that alkaline phosphatase had been produced (FIG. 4), which indicated the differentiation of the cartilage.

7. Transplantation of Chondrocyte Tissue

After removing the medium from the flask, the tissue containing the chondrocytes forming a confluent aggregate was harvested by sucking it with a syringe. Next, the chondrocyte tissue was mixed with fibrin (We preferred autologous fibrin) and the mixture was subcutaneously injected into a defect in a cartilage in a living body.

8. Tissue Culture of Chondrocyte Tissue

The chondrocyte tissue obtained by the above cell culture may be further subjected to tissue culture before transplantation to form a cartilage block. The tissue culture can be carried out either in vitro or in vivo.

In the in vitro tissue culture wherein the chondrocyte tissue obtained by the cell culture is cultured under conditions being close to physiological conditions (for example, by using an artificial body fluid), use can be made of marketed media such as Dulbecco's minimal essential medium (Biological Industries, Beit-Haemek, Israel) or Dulbecco's modified Eagle's medium containing ascorbic acid (50 μg/ml), antibiotics such as penicillin and streptomycin (100 U/ml) and serum (preferably autoserum) (5%). The chondrocyte tissue was introduced into a 6-well plate and cultured in the presence of 5% CO₂ at 37° C. The liquid culture medium was usually replaced every 2 days. It is usually preferred to carry out the culture for 3 weeks, though the culture time may be prolonged or shortened if desired.

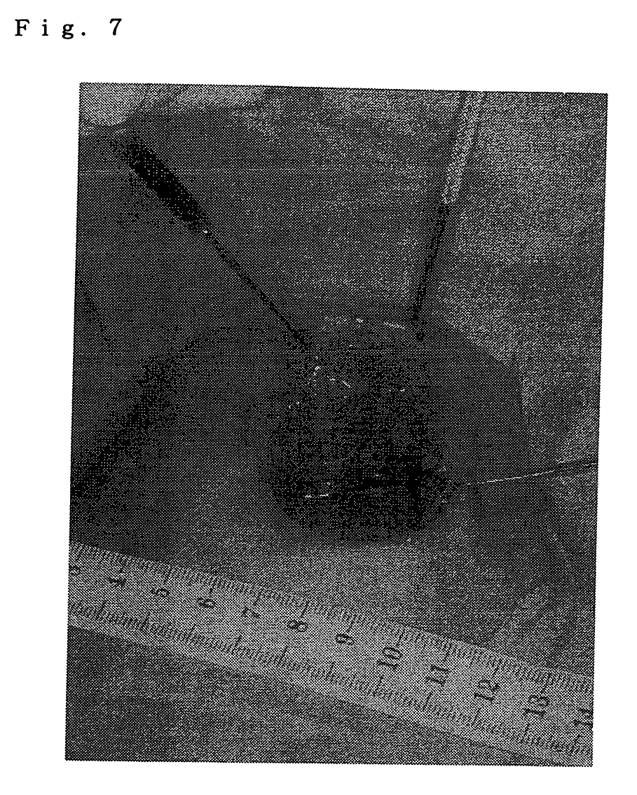

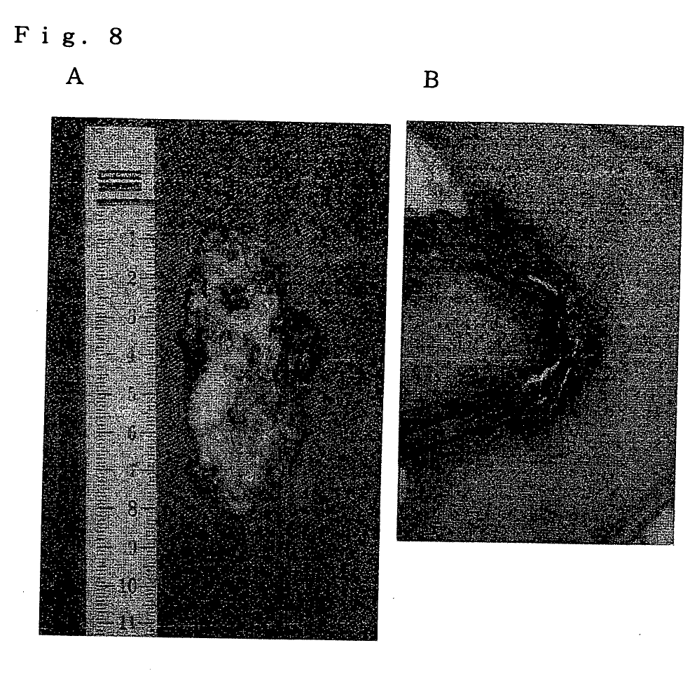

In the in vivo culture, the chondrocyte tissue obtained by the cell culture is mixed with fibrin (preferably autologous fibrin) and then once transplanted into an adequate site (for example, the abdomen wherein a wound is less invisible, being preferred) in a living body to form a cartilage block. Subsequently, it is once taken out from the body and the cartilage shape is modified followed by the transplantation into a target site. This method is favorable in the case of forming a complicated shape such as auricle. Six months after the transplantation, a sample was taken out from the transplanted site and histologically examined. When the sample was stained with hematoxylin-eosine (HE), it was observed that the cells were multilayered and bonded together via the matrix. When the sample was further immunologically stained for type II collagen serving as a molecular marker of chondrocyte tissues, the extracellular matrix was stained, thus indicating that the extracellular matrix was a cartilage-specific matrix (FIG. 5). Moreover, metachromacia was shown in toluidine blue staining, indicating the presence of aggrecan serving as a cartilage marker (FIG. 6). These results suggested that the transplanted chondrocyte tissue had formed a normal cartilage tissue.

9. Practical Transplantation Case

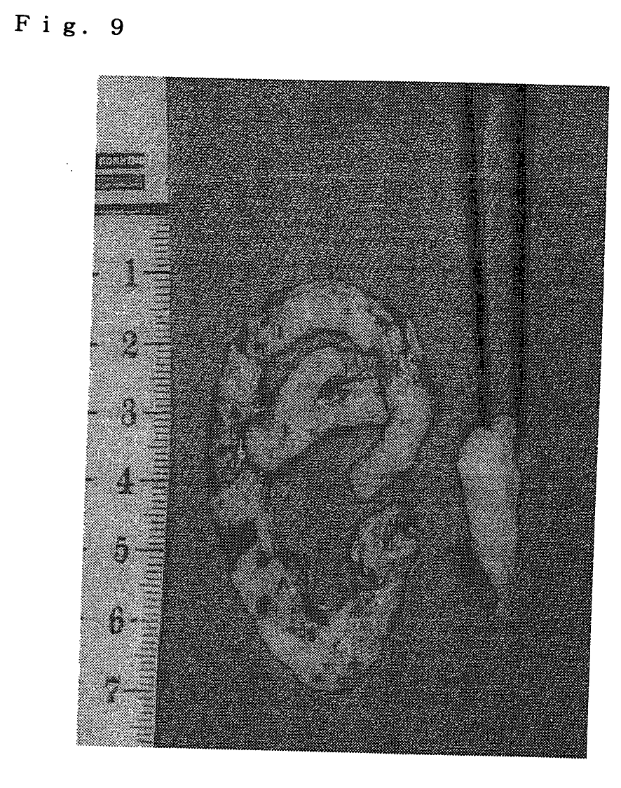

A residual cartilage piece (about 1 cm) was collected from a patient with microtia (congenital ear deformity) and chondrocytes were cultured in the method as described above. After mixing the obtained autologous chondrocyte tissue with autologous fibrin, the mixture was subcutaneously transplanted into the abdomen. Six months thereafter, a cartilage having a sufficient size for forming adult auricle (about 8×4 cm, 1 cm in thickness) was obtained (FIG. 7 and FIGS. 8A and 8B). The obtained cartilage was large enough to form an auricle-shaped graft. Then it was shaped into an auricle-like piece and transplanted to the auricular defect (FIG. 9).

In the existing method of surgically treating microtia, it is required to remove 3 or 4 costal cartilages of an 8 to 10 year-old child, which imposes serious burdens on the donor such as deformation and scar in the chest caused by the removal of cartilages. According to the present invention, these burdens can be minimized. Moreover, it is expected that a graft could be shaped or formed in accordance with the patient's request.

Other embodiments and uses of the invention will be apparent to those skilled in the art from considerations of the specification and practice of the invention disclosed herein. It is intended that the specification and examples be considered exemplary only, with the scope of particular embodiments of the invention indicated by the following claims.

What is claimed is:

1. A cartilage composition for transplantation which contains a chondrocyte tissue per se formed by culturing chondrocytes.

2. A cartilage composition for transplantation containing a cartilage block which is obtained by forming a cartilage tissue by culturing chondrocytes and then subjecting it to tissue culture.

3. The cartilage composition for transplantation as claimed in claim 2, wherein said tissue culture is carried out in vitro.

4. The cartilage composition for transplantation as claimed in claim 2, wherein said tissue culture is carried out in vivo.

5. The cartilage composition for transplantation as claimed in claim 1 or 2, wherein serum is added in said chondrocyte culture.

6. The cartilage composition for transplantation as claimed in claims 1 or 2, wherein autoserum is added in said chondrocyte culture.

7. The cartilage composition for transplantation as claimed in claim 1 or 2, wherein said cartilage composition contains fibrin.

8. The cartilage composition for transplantation as claimed in claim 1 or 2, wherein said cartilage composition contains autologous fibrin.

9. A method of transplanting a cartilage wherein a chondrocyte tissue per se formed by culturing chondrocytes is transplanted.

10. A method of transplanting a cartilage wherein a cartilage block which is obtained by forming a cartilage tissue by culturing chondrocytes and then subjecting it to tissue culture is transplanted.

11. The method of transplanting a cartilage as claimed in claim 10, wherein said tissue culture is carried out in vitro.

12. The method of transplanting a cartilage as claimed in claim 10, wherein said tissue culture is carried out in vivo.

13. The method of transplanting a cartilage as claimed in claim 9 or 10, wherein serum is added in said culturing chondrocytes.

14. The method of transplanting a cartilage as claimed in claim 9 or 10, wherein autoserum is added in said culturing chondrocytes.

15. The method of transplanting a cartilage as claimed in claim 9 or 10, wherein fibrin is added in said transplanting a cartilage.

16. The method of transplanting a cartilage as claimed in claim 9 or 10, wherein autologous fibrin is added in said transplanting a cartilage.
